(19) United States
(12) Patent Application Publication
(10) Pub. No.: US 2019/0054244 A1
(43) Pub. Date: Feb. 21, 2019

(54) IMPEDANCE-BASED DETERMINATION OF WHETHER A HYPODERMIC NEEDLE IS INSERTED INTO THE SKIN

(71) Applicant: QUALCOMM Incorporated, San Diego, CA (US)

(72) Inventors: John Earl AMSCHLER, Del Mar, CA (US); Robert GANTON, San Diego, CA (US); Brian NIZNIK, San Diego, CA (US); Eugene DANTSKER, San Diego, CA (US); Paul Robert HOFFMAN, San Diego, CA (US)

(21) Appl. No.: 16/104,018

(22) Filed: Aug. 16, 2018

Related U.S. Application Data
(60) Provisional application No. 62/547,056, filed on Aug. 17, 2017.

(51) Int. Cl. A61M 5/315

(52) U.S. Cl. CPC A61M 5/3157; A61M 2205/3553; A61M 2205/609; A61M 2205/276; A61M 2205/50; A61M 2230/65; A61M 2205/13; A61M 2205/18

(57) ABSTRACT

Techniques are disclosed for using electrical impedance measurements to help determine whether a hypodermic needle has been inserted into the skin, thereby increasing the accuracy of information provided to the stakeholders and/or enabling other functionality based on the determination. This information can be used and/or combined with other information to help determine whether to administer the drug and/or whether the drug has been administered properly.

### RELATED APPLICATIONS

This application claims the benefit of U.S. Provisional Application No. 62/547,056, filed Aug. 17, 2017, entitled “IMPEDANCE-BASED METHOD TO DETERMINE IF A NEEDLE IS INSERTED IN THE SKIN,” which is assigned to the assignee hereof and incorporated by reference herein in its entirety.

### BACKGROUND

### Background Field

The subject matter disclosed herein relates to hypodermic needles, and more particularly to methods and apparatuses for determining if a hypodermic needle is inserted into the skin.

### Relevant Background

In the field of drug administration, it is often desirable to track drug administration to a patient to help ensure medication compliance, adherence, and persistence by stakeholders, such as the patient him/herself, a healthcare provider, insurance company, etc. This may be especially true in situations where a drug is self-administered by the patient (rather than a healthcare provider). Moreover, even when multiple stakeholders are not involved, it can still be desirable to determine whether a hypodermic needle is being used properly. Problematically, however, traditional devices that deliver drugs via hypodermic needle typically have no capacity of determining whether the drug has been delivered properly.

### SUMMARY

Embodiments disclosed herein use electrical impedance measurement techniques to help determine whether a hypodermic needle has been inserted into the skin, thereby increasing the accuracy of information provided to the stakeholders and/or enabling other functionality based on the determination. This information can be used and/or combined with other information to help determine whether to administer the drug and/or whether the drug has been administered properly.

An example method of administering a drug with a hypodermic needle device, according to the description, comprises obtaining, at the hypodermic needle device, an indication of drug administration by the hypodermic needle device. The method further comprises obtaining, at the hypodermic needle device, one or more impedance measurements during a period of time during which the drug is administered by the hypodermic needle device. The one or more impedance measurements are taken by one or more impedance sensors of the hypodermic needle device, and the one or more impedance measurements are indicative of a location of a needle of the hypodermic needle device, relative to a user's skin, during the period of time. The method further comprises determining an effectiveness of drug administration with the hypodermic needle device based, at least in part, on the one or more impedance measurements.

Embodiments of the method may further comprise one or more of the following features. The one or more impedance sensors may comprise a strain gauge located around a portion of the needle of the hypodermic needle device. The one or more impedance sensors may comprise a strain gauge located between the needle of the hypodermic needle device and at least a portion of a body of the hypodermic needle device. The one or more impedance sensors may measure impedance between an electrode and the needle of the hypodermic needle device. The method may further comprise sending, to another device, an indication of the determination of the effectiveness of the drug administration with the hypodermic needle device. The indication of the determination of the effectiveness of how the drug was administered may comprise an indication that the needle is in tissue, and the other device may comprise a device maintained by a drug adherence or compliance system. The method may further comprise prohibiting an administration of the drug based on the one or more impedance measurements being outside of an acceptable impedance range. The obtaining the one or more impedance measurements may be in response to receiving a triggering event. The one or more impedance measurements may be indicative of a depth the needle of the hypodermic needle device is inserted into tissue.

An example hypodermic needle device, according to the description, comprises one or more impedance sensors configured to take one or more impedance measurements indicative of a location of a needle of the hypodermic needle device, relative to a user's skin, during a period of time in which a drug is administered by the hypodermic needle device, and a processor communicatively coupled to the one or more impedance sensors. The processor is configured to obtain an indication of drug administration by the hypodermic needle device, obtain the one or more impedance measurements, and responsive to the indication of drug administration, determine an effectiveness of drug administration with the hypodermic needle device based at least in part on the one or more impedance measurements.

Embodiments of the hypodermic needle device may comprise one or more of the following features. The one or more impedance sensors may comprise a strain gauge located around a portion of the needle of the hypodermic needle device. The one or more impedance sensors may comprise a strain gauge located between the needle of the hypodermic needle device and at least a portion of a body of the hypodermic needle device. The one or more impedance sensors may be configured to measure impedance between an electrode and the needle of the hypodermic needle device. The hypodermic needle device may further comprise a communication interface configured to send, to another device, an indication of the determination of the effectiveness of the drug administration with the hypodermic needle device. The hypodermic needle device may further comprise a dose-dispensing mechanism, wherein the processor is further configured to cause the dose-dispensing mechanism to prohibit further administration of the drug based on the one or more impedance measurements being outside of an acceptable impedance range.

Another example hypodermic needle device, according to the description, comprises means for obtaining, at the hypodermic needle device, an indication of drug administration by the hypodermic needle device, and means for obtaining one or more impedance measurements during a period of time during which a drug is administered by the hypodermic needle device. The one or more impedance measurements are indicative of a location of a needle of the hypodermic needle device, relative to a user's skin, during the period of time. The hypodermic needle device further comprises means for determining an effectiveness of drug administration with the hypodermic needle device based, at least in part, on the one or more impedance measurements.

Embodiments of the hypodermic needle device may further comprise one or more of the following features. The means for obtaining one or more impedance measurements may comprise a strain gauge located around a portion of the needle of the hypodermic needle device. The means for obtaining one or more impedance measurements may comprise a strain gauge located between the needle of the hypodermic needle device and at least a portion of a body of the hypodermic needle device. The means for obtaining one or more impedance measurements may measure impedance between an electrode and the needle of the hypodermic needle device. The means for sending, to another device, an indication of the determination of the effectiveness of the drug administration with the hypodermic needle device. The hypodermic needle device may further comprise means for generating the indication of the determination of the effectiveness of how the drug was administered such that the indication of the determination of the effectiveness of how the drug was administered comprises an indication that the needle is in tissue. The hypodermic needle device may further comprise means for prohibiting further administration of the drug based on the one or more impedance measurements being outside of an acceptable impedance range. The means for obtaining the one or more impedance measurements may comprise means for obtaining the one or more impedance measurements responsive to receiving a triggering event. The means for obtaining the one or more impedance measurements may comprise means for obtaining the one or more impedance measurements indicative of a depth the needle of the hypodermic needle device is inserted into tissue.

An example non-transitory computer-readable medium, according to the description, has instructions embedded thereon for administering a drug with a hypodermic needle device. The instructions comprise computer code for obtaining, at the hypodermic needle device, an indication of drug administration by the hypodermic needle device, and obtaining, at the hypodermic needle device, one or more impedance measurements during a period of time during which the drug is administered by the hypodermic needle device. The one or more impedance measurements are taken by one or more impedance sensors of the hypodermic needle device, and the one or more impedance measurements are indicative of a location of a needle of the hypodermic needle device, relative to a user's skin, during the period of time. The instructions further comprise computer code for determining an effectiveness of drug administration with the hypodermic needle device based, at least in part, on the one or more impedance measurements.

### BRIEF DESCRIPTION OF DRAWINGS

Non-limiting and non-exhaustive aspects are described with reference to the following figures, wherein like reference numerals refer to like parts throughout the various figures unless otherwise specified.

FIG. 1 is an example system for providing information about the administration of medicine by a hypodermic needle device to one or more stakeholders.

FIG. 2 is an illustration of an example hypodermic needle device, according to an embodiment.

FIG. 3 is a block diagram illustrating the of components of a hypodermic needle device, according to an embodiment.

FIGS. 4A-4C are simplified cross-sectional diagrams of an embodiment of a needle assembly of a hypodermic needle device, illustrating how a needle assembly may be configured to take various types of impedance measurements.

FIG. 5 is a flow diagram illustrating a method of administering a drug with a hypodermic needle device, according to an embodiment.

### DETAILED DESCRIPTION

Several illustrative embodiments will now be described with respect to the accompanying drawings, which form a part hereof. The ensuing description provides embodiment(s) only, and is not intended to limit the scope, applicability or configuration of the disclosure. Rather, the ensuing description of the embodiment(s) will provide those skilled in the art with an enabling description for implementing an embodiment. It is understood that various changes may be made in the function and arrangement of elements without departing from the spirit and scope of this disclosure.

It can be noted that the term “impedance” and variations thereof, as used herein, refer to an electrical impedance. Electrical impedance is a measure of the opposition that an electrical circuit presents to a current when a voltage is applied. As such, the term “impedance” herein also refers to electrical resistance.

Hypodermic needle devices (e.g., injector pens, auto injectors, syringe needles, etc.) administer liquid drugs into the body of a patient (e.g., directly into a muscle, vein, or other location under a patient's skin) by pushing a volume of the drug from a chamber or cylinder within the device through a hypodermic needle that has been injected into the skin of patient. Oftentimes these drugs may be self-administered by the patient, such as when the drugs are administered in an emergency (as may be the case with epinephrine, for example) or frequently administered (as may be the case with insulin, for example).

Establishing that the right dose of the right drug is administered to the right patient at the right time via the right route can be important not only to the person taking the drug, but to many other entities as well. Other stakeholders that have an interest in this information include, for example, the doctor that prescribed and/or is overseeing the administration of the drug, a manufacturer of the drug, an insurance provider (and/or other payer), a government health agency and/or other health organization, and/or the like. For each of the stakeholders, the use and/or misuse of a drug may impact the decisions of a stakeholder with regard to the drug. For example, a drug that is consistently misused may impact whether or how an insurance provider is willing to pay for the use of the drug and/or how to adjust premiums for patients that may consistently misuse drugs. It may also prompt a manufacturer to modify the means of administration of the drug to help reduce the misuse of its administration. All stakeholders may be impacted by use/misuse information in determining how effective a drug for a population of patients.

Embodiments disclosed herein help increase the accuracy of this information provided to the stakeholders by using impedance measurement techniques to determine whether a hypodermic needle has been inserted into the skin. This information can be used and/or combined with other information to help determine whether to administer the drug and/or whether the drug has been administered properly. Additional details are provided herein below.

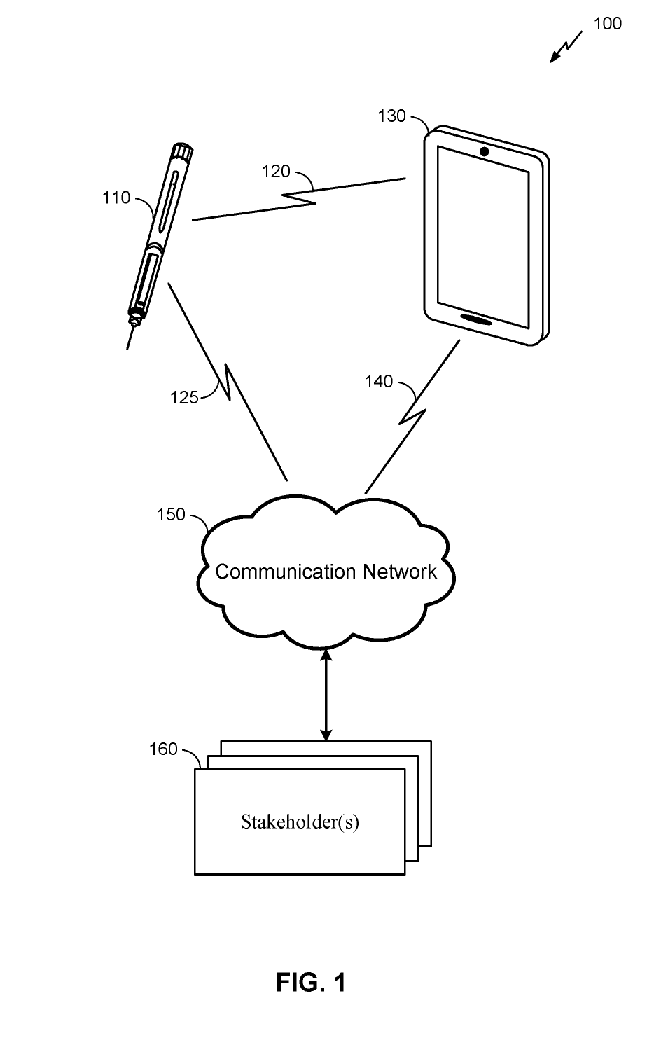

FIG. 1 is an example system 100 for providing information about the administration of medicine by a hypodermic needle device 110 to one or more stakeholders 160. Here, the system 100 may comprise the hypodermic needle device 110 as described herein, along with a connecting device 130, communication network 150, and the stakeholder(s) 160. It will be understood, however, that embodiments of a system 100 may include a different configuration of components, the addition and/or omission of various components, and/or the like, depending on desired functionality. Moreover, it will be understood that techniques described herein may be utilized in a hypodermic needle device 110 that may not necessarily be part of a larger system, such as the system 100 illustrated in FIG. 1.

The hypodermic needle device 110, which is described in more detail herein below, is used to administer a drug to a patient. Here, a person (e.g., a doctor, nurse, or patient him/herself) may administer the drug by engaging a physical mechanism (e.g., pressing down on a plunger, actuating automatic injection, etc.) while a needle of the hypodermic needle device 110 is injected into the patient's skin. In some embodiments, once the drug is administered, the hypodermic needle device 110 can then register, store, and transmit data associated with the administration of the drug to the connecting device 130. This data can be transmitted wirelessly via a communication link 120, using any of a variety of wireless technologies as described in further detail below. That said, some embodiments may additionally or alternatively utilize wired communication.

The connecting device 130 may comprise any of a variety of electronic devices capable of receiving information from the hypodermic needle device 110 and communicating information to the stakeholder(s) 160 via the communication network 150. This can include, for example, a mobile phone, tablet, laptop, portable media player, personal computer, or similar device. In some embodiments, the connecting device 130 may comprise a specialized device utilized for purposes of conveying information from the hypodermic needle device 110 (and possibly other medical devices) to the stakeholder(s) 160. In some embodiments, the connecting device 130 may comprise a device owned and operated by the patient (e.g., the patient's mobile phone). In other embodiments, the connecting device 130 may be owned and/or operated by another entity, such as a healthcare provider, insurance company, government agency, etc.

The connecting device 130 may execute an application to provide the data processing and/or relaying functionality illustrated in FIG. 1. In some embodiments, the application may be configurable by a user, or may simply be downloaded to the connecting device 130 and executed automatically. The application may help establish the communication link 120 between the hypodermic needle device 110 and the connecting device 130, which may or may not require input from the user, depending on desired functionality. In some embodiments, the application may provide instructions to a user on proper use of the hypodermic needle device 110 and/or feedback to a user when improper use of the hypodermic needle device 110 is detected. Additional and/or alternative functionality of an application executed by the connecting device 130 may be utilized as desired. (Such functionality may include simple relaying of the data to a remote destination or interacting with the patient about the drug administration such as confirmation and user feedback.)

The communication network 150 may comprise any of a variety of data communication networks, depending on desired functionality. The communication network 150 can include any combination of radio frequency (RF), optical fiber, satellite, and/or other wireless and/or wired communication technologies. In some embodiments, the communication network 150 can comprise the Internet and/or different data networks may comprise various network types, including cellular networks, Wi-Fi® networks, etc. These types may include, for example, a Code Division Multiple Access (CDMA) network, a Time Division Multiple Access (TDMA) network, a Frequency Division Multiple Access (FDMA) network, an Orthogonal Frequency Division Multiple Access (OFDMA) network, a Single-Carrier Frequency Division Multiple Access (SC-FDMA) network, a WiMax (IEEE 802.16), and so on. A CDMA network may implement one or more radio access technologies (RATs) such as cdma2000, Wideband-CDMA (W-CDMA), and so on. Cdma2000 includes IS-95, IS-2000, and/or IS-856 standards. A TDMA network may implement Global System for Mobile Communications (GSM), Digital Advanced Mobile Phone System (D-AMPS), or some other RAT. An OFDMA network may employ LTE (including LTE category M (CatM) or 5G), LTE Advanced, and so on. LTE, LTE Advanced, GSM, and W-CDMA are described in documents from 3GPP. Cdma2000 is described in documents from a consortium named “3rd Generation Partnership Project 2” (3GPP2). 3GPP and 3GPP2 documents are publicly available. The communication network 150 may additionally or alternatively include a wireless local area network (WLAN), which may also be an IEEE 802.11x network, and a wireless personal area network (WPAN) may be a Bluetooth network, an IEEE 802.15x, Zigbee® network, and/or some other type of network. The techniques described herein may also be used for any combination of wireless wide area network (WWAN), WLAN and/or WPAN.

The communication link 140 between the connecting device 130 and the communication network 150 can vary, depending on the technologies utilized by these components of the system 100. For embodiments where the connecting device 130 is a mobile phone, for example, the communication link 140 may comprise a wireless communication link utilizing the mobile phone's cellular or Wi-Fi® functionality. In embodiments where the connecting device 130 is a personal computer, for example, the communication link 140 may comprise a wired communication link that accesses the communication network 150 via a cable or digital subscriber line (DSL) modem.

It can be noted that some embodiments may not utilize a connecting device 130 to relay data to the communication network 150. In such embodiments, the hypodermic needle device 110 may connect directly to the communication network 150 (as shown in FIG. 1 by communication link 125, which may be used in addition to or as an alternative to communication link 120). For example, the hypodermic needle device 110 may comprise a Long Term Evolution (LTE) category M (Cat-M) device, NarrowBand IoT (NB-IoT), or other Low Power Wide Area Network (LPWAN). Additionally or alternatively, the hypodermic needle device 110 may comprise wireless technology similar to the corresponding functionality of the connecting device 130 described above. In such embodiments, the communication network may additionally or alternatively comprise a Bluetooth Mesh network (such as CSRMesh), a Wi-Fi network, Zigbee, or WWAN (such as LTE, including Cat-M, or 5G). In some embodiments, the hypodermic needle device 110 may connect both with the communication network 150 via communication link 125 and with the connecting device 130 the communication link 120. In such embodiments, the connecting device 130 may not need to separately communicate information regarding the hypodermic needle device 110 to stakeholders 160, but instead the hypodermic needle device 110 may communicate this information directly to the stakeholders 160 via the communication network 150.)

As noted above, the stakeholder(s) 160 may include any of a variety of entities with an interest in the proper administration of medicine by the hypodermic needle device 110. This can include an individual practitioner (e.g., a doctor or nurse), a hospital, a drug manufacturer, an insurance provider (or other payer), a government agency or other health organization, and/or the like. In some embodiments, the user of the hypodermic needle device 110 (e.g., the patient) may also be a stakeholder 160 to which information regarding the use of the hypodermic needle device 110 is provided. Governmental health regulations and/or legal agreements between the patient and/or the stakeholder(s) 160 may apply to the dissemination of information regarding the administration of a drug by the hypodermic needle device 110 to the stakeholder(s) 160. Here, as mentioned above and described in further detail below, the hypodermic needle device 110 can utilize impedance measurement techniques to help ensure the accuracy of the information disseminated to the stakeholder(s) 160. In some embodiments, the stakeholder(s) may maintain a drug adherence or compliant system to track the administration of the drug by the hypodermic needle device 110 and/or administration of other drugs to the patient and/or other patients.

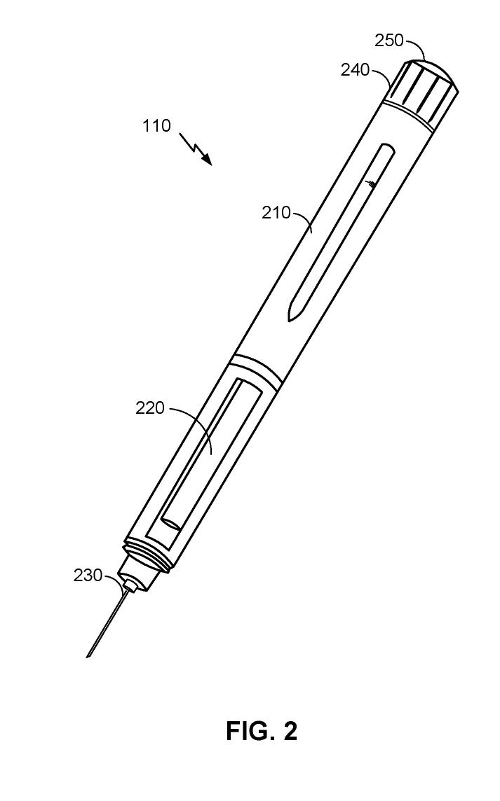

FIG. 2 is an illustration of an example hypodermic needle device 110, according to an embodiment. Here, a body 210 of the hypodermic needle device 110 may house dose dispensing and dose control mechanisms, including electrical and mechanical components, to ensure that a proper dosage of the drug is administered. Mechanical components of a dose dispensing mechanism may include a plunger controlled by the dose control mechanism and configured to displace a volume of the drug through the reservoir chamber 220 and out the needle assembly 230. Embodiments of a hypodermic needle device 110 a dosage dial 240 that can be adjusted (e.g., by turning the dial clockwise or counterclockwise) to alter the dosage amount administered by the hypodermic needle device 110. The dosage may be administered by pressing the button 250, which may be coupled to a dose dispensing mechanism to control the dispensing of the drug.

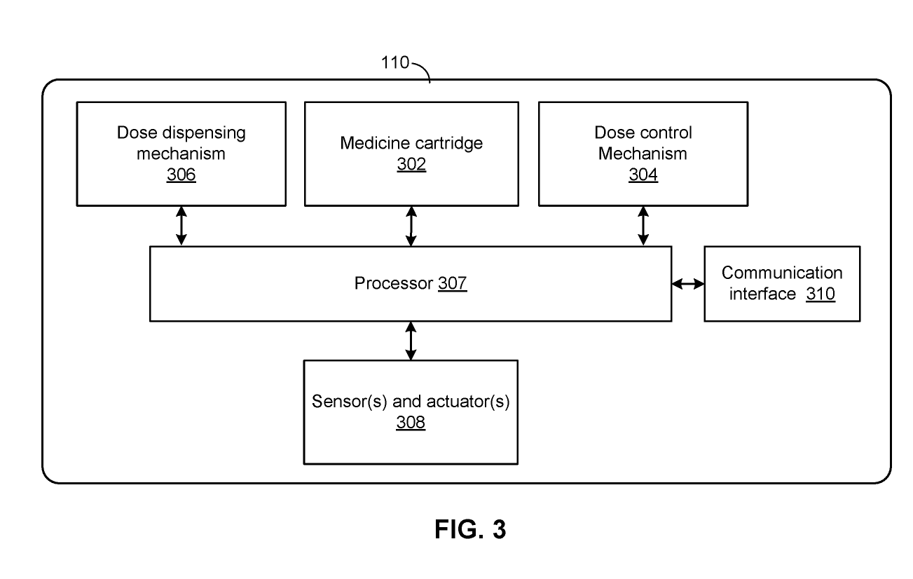

It will be understood however that the hypodermic needle device 110 illustrated in FIG. 2 is provided as a non-limiting example, according to an embodiment. Alternative embodiments may vary in size, shape, and/or other ways. A hypodermic needle device 110 may be described more generally as having various components as illustrated in FIG. 3.

FIG. 3 is a block diagram illustrating the of components of a hypodermic needle device 110, according to an embodiment. The hypodermic needle device 110 can include a housing (not shown) structured to hold a medicine cartridge 302, which may store medicine to be dispensed by the hypodermic needle device 110. The hypodermic needle device 110 can also include a dose control mechanism 304 to select or set a dose of the drug to be dispensed. The hypodermic needle device 110 further includes a dose dispensing mechanism 306 to dispense a dose of the drug, from medicine cartridge 302, based on the dose selected or set by dose control mechanism 304.

The hypodermic needle device 110 may include other devices to facilitate administering of medicine. In the example of FIG. 3, the hypodermic needle device 110 includes sensor(s) and actuator(s) 308. Additionally, the hypodermic needle device 110 can include a processor 307 communicatively coupled with the sensor(s) and actuator(s) 308 and configured to, among other things, control the operations of the actuator(s) based on the information collected by the sensor(s). For example, the sensors of sensor(s) and actuator(s) 308 can collect information of certain physical conditions at, for example, medicine cartridge 302, dose control mechanism 304, and dose dispensing mechanism 306. Based on the collected information, the processor 307 can control the actuators of sensor(s) and actuator(s) 308 to change the operations of dose control mechanism 304 and/or dose dispensing mechanism 306. For example, based on impedance measurement information, which may be obtained from one or more sensors of the sensor(s) and actuator(s) 308, the actuators can be controlled (e.g., by the processor 307) to change the operations of dose control mechanism 304 and/or dose dispensing mechanism 306 to prohibit administration of the drug.

The processor 307 may comprise without limitation one or more general-purpose processors, one or more special-purpose processors (such as digital signal processing (DSP) chips, graphics acceleration processors, application specific integrated circuits (ASICs), and/or the like), and/or other processing structure or means, which can be configured to perform one or more of the methods described herein. To help increase shelf life of the hypodermic needle device 110, the processing unit may be configured to operate in an extremely low power mode that, along with the capacity of a power supply (not shown), can allow the electrical components of the hypodermic needle device 110to be used in after a substantially long time of no use. In some embodiments, the processor 307 may utilize additional hardware and/or software components (e.g., a memory) to provide the functionality described herein.

The hypodermic needle device 110 may include a communication interface 310 which can communicate using wireless and/or wired means (e.g., via communication link 120 and/or 125 of FIG. 1). Communication interface 310 may enable transmission of information related to administering the drug, including an impedance measurement and/or an indication of whether the drug was administered properly. Additionally or alternatively, the hypodermic needle device 110 may communicate information related to a quantity of medicine to be dispensed, a quantity of medicine that has been dispensed, a quantity of medicine remaining in medicine cartridge 302, etc. The information can then be displayed to the user via an user interface, to assist the user in administering of the medicine.

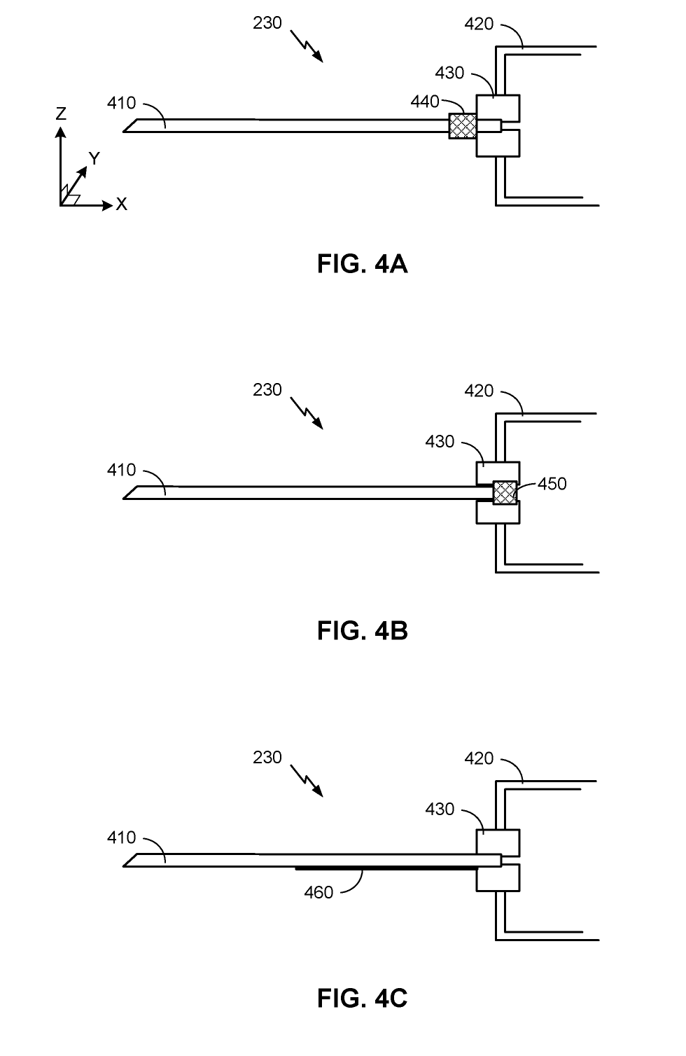

FIGS. 4A-4C are simplified cross-sectional diagrams of an embodiment of a needle assembly 230 of a hypodermic needle device 110, illustrating how a needle assembly 230 may be configured to take various types of impedance measurements. These cross-sectional diagrams illustrate the needle 410 (a hollow tube typically fashioned from stainless steel) secured to the body 420 of the hypodermic needle device 110 by a securing portion 430. According to some embodiments, the securing portion 430 may be formed from molded plastic. It will be appreciated, however, that the impedance measurement techniques illustrated herein may be utilized by various types of hypodermic needle devices, which may have different components than those illustrated in FIGS. 4A-4C (which are simplified drawings provided for illustrative purposes).

In FIG. 4A, a ring-shaped strain gauge 440 is located around a portion of the needle 410 and used to provide impedance measurements. (It will be appreciated that the strain gauge 440 may have any of a variety of thicknesses and lengths, and may be located at different portions of the needle 410 than illustrated in FIG. 4A, depending on desired functionality.) The strain gauge 440 may be electrically connected (e.g., via wires, not shown) with circuitry configured to detect various types of strain on the strain gauge 410, including movement of the needle 410 in any or all of the X, Y, or Z axes. The strain gauge may be calibrated during manufacture and/or prior to use. During proper use, when the needle 410 is inserted into a patient's skin, the insertion can cause movement of the needle 410 which, in turn, can cause the strain gauge 410 to register a different impedance. This impedance can be detected and recorded by circuitry (analog and/or digital) in the hypodermic needle device 110, which can interpret the change of impedance as possible insertion of the needle 410 into the patient's skin. According to some embodiments, this change of impedance may be within a threshold or range. (If a change in impedance is outside the range or threshold, it may be interpreted as possible misuse of the hypodermic needle device 110.)

The circuitry in the hypodermic needle device 110 can measure the impedance of the strain gauge 440 in any of a variety of ways, depending on desired functionality. In some embodiments, the hypodermic needle device 110 may obtain an indication of the administration of the drug by the hypodermic needle device 110 from one or more sensors or actuators (e.g., sensor(s) and actuator(s) 308), including a button (e.g., button 250) pressed by the user to actuate administration of the drug. According to some embodiments, the indication of the administration of the drug may trigger the circuitry of the hypodermic needle device 110 to measure the impedance of the strain gauge 440. If the strain gauge measures a certain change of impedance within a threshold amount of time of receiving the indication of the administration of the drug, then the hypodermic needle device 110 may determine that the needle 410 was in the patient's skin during the time of administration. For example, an initial impedance measurement may be taken when the hypodermic needle device 110 obtains an indication of the administration of the drug by the hypodermic needle device 110. One or more additional impedance measurements may be taken over the course of a period of time following the indication of the administration of the drug to then determine whether a threshold change of impedance occurred within a threshold amount of time. Impedance values and/or time values for these thresholds may vary, depending on strain gauge 440 output, desired functionality, and/or other factors. According to some embodiments, this information may be combined with additional information to determine whether the drug was administered properly by the hypodermic needle device 110.

In FIG. 4B, a strain gauge 450 is located between the needle 410 and the securing portion 430 of the hypodermic needle device 110. The functionality here may be similar to FIG. 4A, although (depending on how restrictive the securing portion 430 may be on the detected movement of the needle 410 by the strain gauge 450) the strain gauge 450 may detect fewer types of movement of the needle 410. In particular, the strain gauge 450 may detect longitudinal movement along the X axis more readily than movement in the Y or Z axes. Nonetheless, longitudinal movement along the x-axis might be more indicative of proper insertion of the needle 410 into the patient's skin.

Additionally or alternatively, embodiments may use electrodes to detect impedance of the body of the patient by measuring electrical impedance between the electrodes. For example, FIG. 4C illustrate how an electrode 460 is located on the side of the needle 410 such that, when the needle 410 is inserted into the patient's skin, the electrode 460 will come in contact with at least a portion of the patient's body. To prevent the electrode 460 from coming into electrical contact with the needle 410, the needle 410 may be coated with an insulating material (not shown) to electrically insulate the electrode 460 from the needle 410. This can enable the needle 410 to serve as a second electrode. Wires (not shown) may electrically connect the electrodes to circuitry to detect electrical impedance between the electrodes.

It will be understood, however, that alternative embodiments may utilize more than two electrodes, and/or utilize a different configuration of electrodes than illustrated in FIG. 4C. in some embodiments, for example, the needle 410 may serve as an electrode, and another electrode may be located elsewhere on the hypodermic needle device that comes in contact with a patient's skin during proper self-administration (e.g., the plunger or button used to administer the drug). Thus, an impedance measurement may comprise measuring an impedance of the patient's body from one electrode (e.g., the needle 410) to another electrode (e.g., an electrode on the plunger, button, and/or elsewhere on the hypodermic needle device). Depending on desired functionality, other surfaces of the hypodermic deal device may include one or more other electrodes to take such impedance measurements.

An impedance measurement may be triggered by and/or used in a manner similar to the manner described above with regard to FIG S. 4A-4B. Here, however, the impedance measurement can be a measurement of impedance between the needle 410 and the electrode 460 by, for example, setting a relatively small voltage between the needle 410 and the electrode 460 and measuring any current. If the needle is not inserted into the patient's skin, but surrounded by air, detected impedance will be extremely high. However, if the needle is properly inserted into the patient's skin to the point where the electrode 460 comes in contact with the patient's body, much lower impedance will be measured. They can then be determined that the needle 410 has been properly inserted into a patient's skin if the measured impedance is less than a maximum threshold amount (e.g., a maximum expected impedance of the patient's body), or within a range of expected impedance values.

In the above embodiments, one or more impedance measurements may be taken, based on any of a variety of events, and depending on various factors such as available battery life. For example, in some embodiments, as described above, impedance measurements may be taken based on a triggering event, such as an indication that a drug is being and/or is about to be administered. (A triggering event that a drug is being and/or is about to be administered may comprise, for example, pressing button 250 to administer the drug, pressing a separate button (e.g., an “on” button) or activating another input device to power the device or awake the device from a low power state, taking a lid off of the device to expose the needle (in embodiments in which the device includes a sensor that can detect when the lid has been removed) or the like.) In other embodiments, impedance measurements may be taken periodically and/or based on a schedule.

As noted above, impedance measurements indicative of the needle 410 being in the patient's skin can be used in various ways. For example, as noted above, an impedance measurement may be used to determine whether the drug was administered effectively. (That is, if the impedance measurement was indicative that the needle 410 was inserted into the patient's skin during administration of the drug, the impedance measurement can be interpreted as an indicator that the drug was administered effectively. Alternatively, if the impedance measurement was indicative that the needle 410 was not inserted into the patient's skin during administration of the drug, the impedance measurement can be interpreted as an indicator that the drug was not administered effectively.) In such embodiments, the impedance measurement may be combined with one or more other types of data (e.g., sensor data indicating that the drug has been administered) to make the determination of effectiveness. This determination and/or the underlying impedance measurement(s) may be sent to another device, such as a connecting device (e.g., connecting device 130 of FIG. 1) and/or a device of one or more stakeholders (e.g., stakeholder(s) 160).

Additionally or alternatively, one or more impedance measurements may be used to determine whether or not to administer the drug in the first place. For instance, if an impedance measurement is not indicative that the needle of the hypodermic needle device has been inserted properly into a patient's skin, then the hypodermic needle device may prevent administration of the drug. That is, it may prevent one or more actuators from administering the drug and/or activate a lock that prohibits use of the hypodermic needle device.

In some embodiments, additional verification may be used to prevent accidental or improper drug administration. For example, in some embodiments, the plunger or button used to administer the drug may have an additional verification element (such as an electrode, skin sensor, fingerprint sensor, etc.), and the hypodermic needle device may prevent administration of the drug if, in addition to receiving an impedance measurement indicative of the needle being inserted into the patient's skin, the additional verification element in the plunger provides additional verification (e.g., verifies an accurate fingerprint, that the plunger is being touched by skin) etc. In some embodiments, the additional verification element may be located elsewhere on the medical device (other than the plunger or button used to administer the drug).

When used with techniques that prevent administration of the drug unless proper verification is first made, these techniques can help prevent misuse of the drug. For instance, as indicated above, impedance measurements may help ensure that the drug is self-administered (e.g., by taking an impedance measurement between the needle and an electrode on the plunger of the hypodermic needle device). In some instances, another person may be authorized to administer the drug for the patient (and, in fact, the patient may not be authorized to self-administer), in which fingerprint verification using a sensor on the plunger (or elsewhere, either on the hypodermic needle device during communication with the hypodermic needle device) can be used with the impedance measurement techniques described herein to help ensure proper administration of the drug by the proper administrator.

In some embodiments, the impedance measurement may be used for other types of verification. For example, in some embodiments, the impedance measurement may be able to identify one or more characteristics of the pulse of a person into which the needle has been injected. These characteristics may include a pulse shape and/or frequency, for example. These one or more characteristics may be used to identify the potential age of the user and/or strain on the user. This additional verification may be used to prevent administration (e.g., until the strain on the user has gone down on) and/or provide additional information, such as send out an alert if the impedance measurement indicates an age outside the range of the user. In some embodiments, the display of a connected device (e.g., connected device 130 of FIG. 1) may be used to show the alert and/or reasoning why verification failed. In some embodiments, the alert on the display may be accompanied and/or replaced with a sound and/or vibration. Additionally or alternatively, the hypodermic needle device itself may have one or more light-emitting diodes (LEDs), an audio speaker, e-paper display, and/or other output component capable of providing an indication that verification has failed.

In some embodiments, verification can be made using one or more connected devices. For example, in some embodiments a connected device may measure a patient's impedance via electrodes, which may be utilized as an identifying feature of the patient. This verification may be valid for a certain period of time, and the impedance and/or period of time may be conveyed from the connected device to the hypodermic needle device. The hypodermic needle device may then take a separate impedance measurement of the patient to determine whether it matches the impedance received from the connecting device (within a threshold degree of variation, which may vary, depending on the types of electrodes used in the hypodermic needle device and/or connected device), and (optionally) whether the certain period of time is not yet elapsed. The hypodermic needle device may then allow administration of the drug if the impedance matches and (optionally) the certain period of time is not yet elapsed. Otherwise, the hypodermic needle device may prevent administration of the drug.

In some embodiments, the connected device may be used to provide non-impedance-based verification (e.g., fingerprint scan, iris scan, facial recognition, etc.), if so equipped. After such verification (and optionally within a predetermined amount of time after verification), the hypodermic needle device may then administer the drug based on a determination that the needle has been inserted into skin (e.g., an impedance measurement). Verification using the connected device may be enabled via a software application executed by the connected device.

Additionally or alternatively, according to some embodiments, an electrical impedance measurement may be correlated to a depth of the needle in the tissue; different depths corresponding to different impedance values. Embodiments may use this information in any of a variety of ways, including, (1) notifying a user if the needle is too shallow or too deep (train over time), (2) locking a drug delivery mechanism or otherwise preventing administration of the drug until proper needle depth is achieved, and/or (3) recognizing different needle injection locations on the user (e.g., arm, leg, buttocks, etc.) and/or type of tissue into which the needle was injected (e.g., skin, fat, muscle, etc.) based on different impedance results—and (optionally) notifying a user and/or preventing drug administration if the use is not in a “correct” location and/or tissue type (as identified by the measured impedance). Calibration for each specific individual could pinpoint those and improve accuracy. As such, embodiments may utilize calibration and absolute determination of the body location, and/or embodiments may confirm that a series of injections was made in the same location on a given individual. Again, notifications to a user may be provided by a connected device (e.g. via sound, a display, etc.) and/or by the hypodermic needle device itself (e.g., via sound, e-paper display, one or more LEDs, etc.).

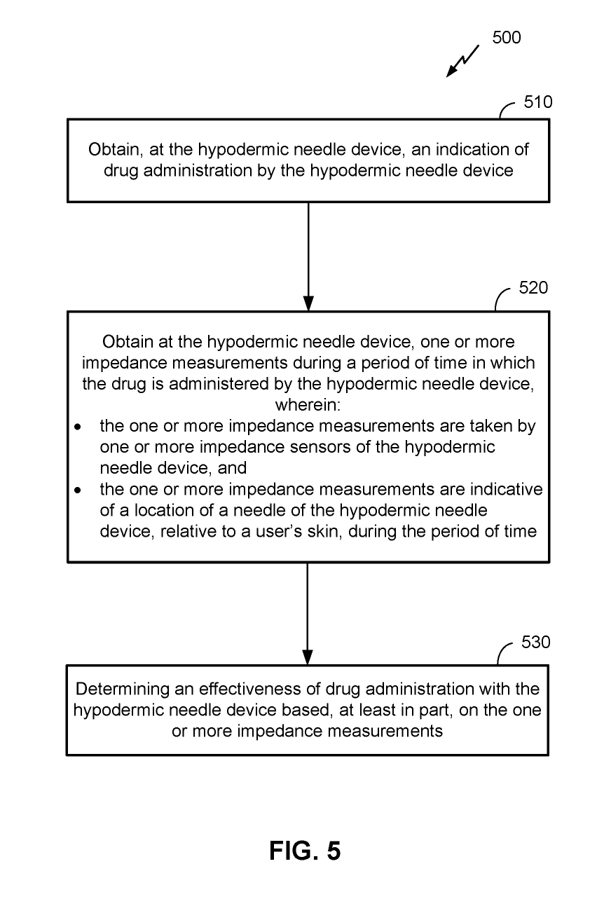

FIG. 5 is a flow diagram illustrating a method 500 of administering a drug with a hypodermic needle device, according to an embodiment. It can be noted that, as with figures appended hereto, FIG. 5 is provided as a non-limiting example. Other embodiments may vary, depending on desired functionality. For example, the functional blocks illustrated in method 500 may be combined, separated, or rearranged to accommodate different embodiments. The method 500 may be performed by a hypodermic needle device. Means for performing the functionality of method 500 may include one or more components of the hypodermic needle device, including hardware and/or software components, as illustrated in FIG. 3 above. Hardware components may include analog and/or digital circuitry, including a processor (e.g., processor 307 of FIG. 3). A person of ordinary skill in the art will appreciate the various means by which the functions in method 500 may be performed.

At block 510, an indication of drug administration by the hypodermic needle device is obtained at the hypodermic needle device. Here, the indication may be, for example, that the hypodermic needle device will soon administer the drug, is currently administering the drug, or has recently administered the drug. This information, may be obtained for example, by a processor from one or more sensors. For instance, a sensor may be coupled with the mechanism for administering the drug (e.g., a plunger or button), detecting physical movement, sound, pressure, fluid flow, and/or other characteristic(s) indicating future, current, and/or recent drug administration. In some embodiments, the removal of a lid (e.g., over the needle), the press of a power (or similar) button, or other action may indicate that the hypodermic needle device may soon administer the drug. The hypodermic needle device may have sensors or other components capable of detecting these actions. In some embodiments, the method may include releasing a locking mechanism of the hypodermic needle device to enable administration of the drug prior to the functionality at block 510, in response to obtaining verification information, as described in the embodiments above, for example. Means for performing the functionality of block 510 may comprise, for example, a processor 307, sensor(s) and actuator(s) 308, a dose dispensing mechanism 306, and/or other components of the hypodermic needle device 110 illustrated in FIG. 3 and described above.

The functionality at block 520 comprises obtaining, at the hypodermic needle device, one or more impedance measurements during a period of time during which the drug is administered by the hypodermic needle device. Here, the one or more impedance measurements are taken by one or more impedance sensors of the hypodermic needle device, and tone or more impedance measurements are indicative of a location of a needle, relative to a user's scan, of the hypodermic needle device during the period of time. As indicated above, the period of time may comprise a period of time before, during, and/or after a time at which the drug is administered by the hypodermic needle device. In some embodiments, the one or more impedance sensors comprise a strain gauge located around a portion of the needle of the hypodermic needle device, a strain gauge located between the needle of the hypodermic device and at least a portion of the hypodermic needle device, and/or using an electrode coupled with the needle of the hypodermic needle device. In the latter case, impedance measurement may be made by measuring an impedance between the electrode and the needle.

In some embodiments, the obtaining the one or more impedance measurements is in response to receiving a triggering event. As previously indicated, the triggering event may comprise the pressing of a button of the hypodermic needle device, the activation of an actuator, sensor data indicating the administration of the drug, etc., and/or may include the functionality of block 510. Means for performing the functionality of block 520 may comprise, for example, a processor 307, the sensor(s) and actuator(s) 308 (which can include the one or more impedance sensors), and/or other components of the hypodermic needle device 110 illustrated in FIG. 3 and described above.

At block 530, the functionality comprises determining an effectiveness of the drug administration by the hypodermic needle device based, at least in part, on the one or more impedance measurements. As indicated above, an impedance measurement may indicate that the needle has been inserted into a user's skin. Moreover, as previously discussed, impedance may further indicate how deep the needle has been injected into the skin of a user, where, on the body of the user, the needle has been injected, and/or the like. As such, these measurements can inform a determination of the effectiveness of how the drug was administered. In some embodiments, a change of impedance from a previously-measured impedance may comprise an indication that the needle has been injected into the skin of a user. In some embodiments, the impedance measurement may fall within a range for threshold indicating insertion of the needle into the patient's skin. Means for performing the functionality of block 530 may comprise, for example, the processor 307, sensor(s) and actuator(s) 308, and/or other components of the hypodermic needle device 110 illustrated in FIG. 3 and described above.

Additional functions may be performed, depending on desired functionality. For instance, some embodiments may further include sending, to another device, an indication of the determination of the effectiveness of how the drug was administered with the hypodermic needle device. Some embodiments may further include receiving one or more additional impedance measurements and prohibiting a second administration of the drug based on the one or more additional impedance measurements.

It will be apparent to those skilled in the art that substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

With reference to the appended figures, components that may comprise memory may comprise non-transitory machine-readable media. The term “machine-readable medium” and “computer-readable medium” as used herein, refer to any storage medium that participates in providing data that causes a machine to operate in a specific fashion. In embodiments provided hereinabove, various machine-readable media might be involved in providing instructions/code to processing units and/or other device(s) for execution. Additionally or alternatively, the machine-readable media might be used to store and/or carry such instructions/code. In many implementations, a computer-readable medium is a physical and/or tangible storage medium. Such a medium may take many forms, including but not limited to, non-volatile media, volatile media, and transmission media. Common forms of computer-readable media include, for example, magnetic and/or optical media, any other physical medium with patterns of holes, a RAM, a PROM, EPROM, a FLASH-EPROM, any other memory chip or cartridge, a carrier wave as described hereinafter, or any other medium from which a computer can read instructions and/or code.

The methods, systems, and devices discussed herein are examples. Various embodiments may omit, substitute, or add various procedures or components as appropriate. For instance, features described with respect to certain embodiments may be combined in various other embodiments. Different aspects and elements of the embodiments may be combined in a similar manner. The various components of the figures provided herein can be embodied in hardware and/or software. Also, technology evolves and, thus, many of the elements are examples that do not limit the scope of the disclosure to those specific examples.

Reference throughout this specification to “one example”, “an example”, “certain examples”, or “exemplary implementation” means that a particular feature, structure, or characteristic described in connection with the feature and/or example may be included in at least one feature and/or example of claimed subject matter. Thus, the appearances of the phrase “in one example”, “an example”, “in certain examples” or “in certain implementations” or other like phrases in various places throughout this specification are not necessarily all referring to the same feature, example, and/or limitation. Furthermore, the particular features, structures, or characteristics may be combined in one or more examples and/or features.

Some portions of the detailed description included herein are presented in terms of algorithms or symbolic representations of operations on binary digital signals stored within a memory of a specific apparatus or special purpose computing device or platform. In the context of this particular specification, the term specific apparatus or the like includes a general purpose computer once it is programmed to perform particular operations pursuant to instructions from program software. Algorithmic descriptions or symbolic representations are examples of techniques used by those of ordinary skill in the signal processing or related arts to convey the substance of their work to others skilled in the art. An algorithm is here, and generally, is considered to be a self-consistent sequence of operations or similar signal processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has proven convenient at times, principally for reasons of common usage, to refer to such signals as bits, data, values, elements, symbols, characters, terms, numbers, numerals, or the like. It should be understood, however, that all of these or similar terms are to be associated with appropriate physical quantities and are merely convenient labels. Unless specifically stated otherwise, as apparent from the discussion herein, it is appreciated that throughout this specification discussions utilizing terms such as “processing,” “computing,” “calculating,” “determining” or the like refer to actions or processes of a specific apparatus, such as a special purpose computer, special purpose computing apparatus or a similar special purpose electronic computing device. In the context of this specification, therefore, a special purpose computer or a similar special purpose electronic computing device is capable of manipulating or transforming signals, typically represented as physical electronic or magnetic quantities within memories, registers, or other information storage devices, transmission devices, or display devices of the special purpose computer or similar special purpose electronic computing device.

The terms, “and”, “or”, and “and/or” as used herein may include a variety of meanings that also are expected to depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein may be used to describe any feature, structure, or characteristic in the singular or may be used to describe a plurality or some other combination of features, structures or characteristics. Though, it should be noted that this is merely an illustrative example and claimed subject matter is not limited to this example.

Therefore, it is intended that claimed subject matter not be limited to the particular examples disclosed, but that such claimed subject matter may also include all aspects falling within the scope of appended claims, and equivalents thereof.

What is claimed is:

1. A method of administering a drug with a hypodermic needle device, the method comprising:
obtaining, at the hypodermic needle device, an indication of drug administration by the hypodermic needle device;
obtaining, at the hypodermic needle device, one or more impedance measurements during a period of time during which the drug is administered by the hypodermic needle device, wherein:
the one or more impedance measurements are taken by one or more impedance sensors of the hypodermic needle device, and
the one or more impedance measurements are indicative of a location of a needle of the hypodermic needle device, relative to a user's skin, during the period of time; and
determining an effectiveness of drug administration with the hypodermic needle device based, at least in part, on the one or more impedance measurements.

2. The method of claim 1, wherein the one or more impedance sensors comprise a strain gauge located around a portion of the needle of the hypodermic needle device.

3. The method of claim 1, wherein the one or more impedance sensors comprise a strain gauge located between the needle of the hypodermic needle device and at least a portion of a body of the hypodermic needle device.

4. The method of claim 1, wherein the one or more impedance sensors measures impedance between an electrode and the needle of the hypodermic needle device.

5. The method of claim 1, further comprising sending, to another device, an indication of the determination of the effectiveness of the drug administration with the hypodermic needle device.

6. The method of claim 5, wherein:
the indication of the determination of the effectiveness of how the drug was administered comprises an indication that the needle is in tissue, and
the other device comprises a device maintained by a drug adherence or compliance system.

7. The method of claim 1, further comprising prohibiting an administration of the drug based on the one or more impedance measurements being outside of an acceptable impedance range.

8. The method of claim 1, wherein the obtaining the one or more impedance measurements is in response to receiving a triggering event.

9. The method of claim 1, wherein the one or more impedance measurements are indicative of a depth the needle of the hypodermic needle device is inserted into tissue.

10. A hypodermic needle device, comprising:
one or more impedance sensors configured to take one or more impedance measurements indicative of a location of a needle of the hypodermic needle device, relative to a user's skin, during a period of time in which a drug is administered by the hypodermic needle device; and
a processor communicatively coupled to the one or more impedance sensors and configured to:
obtain an indication of drug administration by the hypodermic needle device;
obtain the one or more impedance measurements; and
responsive to the indication of drug administration, determine an effectiveness of drug administration with the hypodermic needle device based at least in part on the one or more impedance measurements.

11. The hypodermic needle device of claim 10, wherein the one or more impedance sensors comprise a strain gauge located around a portion of the needle of the hypodermic needle device.

12. The hypodermic needle device of claim 10, wherein the one or more impedance sensors comprise a strain gauge located between the needle of the hypodermic needle device and at least a portion of a body of the hypodermic needle device.

13. The hypodermic needle device of claim 10, wherein the one or more impedance sensors are configured to measure impedance between an electrode and the needle of the hypodermic needle device.

14. The hypodermic needle device of claim 10, further comprising a communication interface configured to send, to another device, an indication of the determination of the effectiveness of the drug administration with the hypodermic needle device.

15. The hypodermic needle device of claim 10, further comprising a dose-dispensing mechanism, wherein the processor is further configured to cause the dose-dispensing mechanism to prohibit further administration of the drug based on the one or more impedance measurements being outside of an acceptable impedance range.

16. A hypodermic needle device comprising:
means for obtaining, at the hypodermic needle device, an indication of drug administration by the hypodermic needle device;
means for obtaining one or more impedance measurements during a period of time during which a drug is administered by the hypodermic needle device, wherein the one or more impedance measurements are indicative of a location of a needle of the hypodermic needle device, relative to a user's skin, during the period of time; and
means for determining an effectiveness of drug administration with the hypodermic needle device based, at least in part, on the one or more impedance measurements.

17. The hypodermic needle device of 16, wherein the means for obtaining one or more impedance measurements comprises a strain gauge located around a portion of the needle of the hypodermic needle device.

18. The hypodermic needle device of 16, wherein the means for obtaining one or more impedance measurements comprises a strain gauge located between the needle of the hypodermic needle device and at least a portion of a body of the hypodermic needle device.

19. The hypodermic needle device of 16, the means for obtaining one or more impedance measurements measures impedance between an electrode and the needle of the hypodermic needle device.

20. The hypodermic needle device of 16, further comprising means for sending, to another device, an indication of the determination of the effectiveness of the drug administration with the hypodermic needle device.

21. The hypodermic needle device of 20, further comprising means for generating the indication of the determination of the effectiveness of how the drug was administered such that the indication of the determination of the effectiveness of how the drug was administered comprises an indication that the needle is in tissue.

22. The hypodermic needle device of 16, further comprising means for prohibiting further administration of the drug based on the one or more impedance measurements being outside of an acceptable impedance range.

23. The hypodermic needle device of 16, wherein the means for obtaining the one or more impedance measurements comprises means for obtaining the one or more impedance measurements responsive to receiving a triggering event.

24. The hypodermic needle device of 16, wherein the means for obtaining the one or more impedance measurements comprise means for obtaining the one or more impedance measurements indicative of a depth the needle of the hypodermic needle device is inserted into tissue.

25. A non-transitory computer-readable medium having instructions embedded thereon for administering a drug with a hypodermic needle device, the instructions comprising computer code for:
obtaining, at the hypodermic needle device, an indication of drug administration by the hypodermic needle device;
obtaining, at the hypodermic needle device, one or more impedance measurements during a period of time during which the drug is administered by the hypodermic needle device, wherein:
the one or more impedance measurements are taken by one or more impedance sensors of the hypodermic needle device, and
the one or more impedance measurements are indicative of a location of a needle of the hypodermic needle device, relative to a user's skin, during the period of time; and
determining an effectiveness of drug administration with the hypodermic needle device based, at least in part, on the one or more impedance measurements.
